(19) United States
(12) Patent Application Publication
(10) Pub. No.: US 2026/0325592 A1
(43) Pub. Date: Oct. 22, 2026

(54) SIX-STRAIN PROBIOTIC FOR PREVENTING SEVERE HEATSTROKE AND USE THEREOF

(71) Applicants: Zhejiang Chinese Medical University, Zhejiang (CN); Hangzhou Negative Entropy Health Technology Co., LTD., Zhejiang (CN)

(72) Inventors: Cuiqing Liu, Zhejiang (CN); Wenjun Fan, Zhejiang (CN); Xuan Zhu, Zhejiang (CN); Shasha Xiang, Zhejiang (CN); Wenbin Zhao, Zhejiang (CN); Yiyuan Ye, Zhejiang (CN); Tong Hou, Zhejiang (CN); Ran Li, Zhejiang (CN)

(73) Assignees: Zhejiang Chinese Medical University, Zhejiang (CN); Hangzhou Negative Entropy Health Technology Co., LTD., Zhejiang (CN)

(21) Appl. No.: 19/649,090

(22) Filed: Apr. 15, 2026

(30) Foreign Application Priority Data
Apr. 21, 2025 (CN) 202510499155.2

(51) Int. Cl. A61K 35/747; A61K 35/742; A61K 35/745; A61P 1/00; A61P 43/00; A61K 35/00

(52) U.S. Cl. CPC A61K 35/747; A61K 35/742; A61K 35/745; A61P 1/00; A61P 43/00; A61K 2035/115

(57) ABSTRACT

Disclosed herein are a six-strain probiotic for preventing severe heatstroke and use thereof, the probiotic composition including Bacillus coagulans XY2, Lactobacillus plantarum KL100, Lactobacillus rhamnosus TR08, Lactobacillus helveticus TR05, Lactobacillus casei TR01, and Bifidobacterium longum TR17. New uses of the probiotic composition in preventing severe heatstroke, heatstroke complicated by liver injury, heatstroke complicated by lung injury, and heatstroke complicated by myocardial injury, and in enhancing intestinal mucosal barrier function are disclosed. The six-strain probiotic preparation of this disclosure can effectively protect the body's condition, enhance the body's resistance to high temperatures, and maintain the balance of the body's thermoregulatory mechanisms, thereby preventing or alleviating the occurrence of severe heatstroke in the body, and has the effects of preventing severe heatstroke and alleviating heatstroke complicated by liver injury, lung injury, and myocardial injury.

### CROSS-REFERENCE TO RELATED APPLICATION

This application claims the priority benefit of China application serial no. 202510499155.2, filed on Apr. 21, 2025. The entirety of the above-mentioned patent application is hereby incorporated by reference herein and made a part of this specification.

### TECHNICAL FIELD

This disclosure relates to the technical field of biomedicine and, in particular, to use of a six-strain probiotic in preparation of a drug for preventing severe heatstroke, heatstroke complicated by liver injury, heatstroke complicated by lung injury, and heatstroke complicated by myocardial injury, and for enhancing intestinal mucosal barrier function.

### BACKGROUND

Heatstroke caused by extreme high temperatures is a febrile illness characterized by a rapid increase in core body temperature exceeding 40°C, often accompanied by central nervous system dysfunction (such as confusion, seizure, or coma) and damage to multiple organs such as liver and intestine, resulting from a complex interaction between heat-related cytotoxicity, inflammatory response, and coagulation disorders. Heatstroke can be classified into prodromal heatstroke, mild heatstroke, and severe heatstroke. Prodromal heatstroke and mild heatstroke are relatively easy to recover from in a short time after leaving the high-temperature environment, while severe heatstroke is often accompanied by multiple organ dysfunction syndrome, which can easily lead to organ damage or even death. In recent years, occupational severe heatstroke cases have been frequently reported in China, all of which were severe heatstroke caused by outdoor work or indoor work without adequate cooling facilities during high-temperature weather. Therefore, how to better and more effectively prevent heatstroke, especially severe heatstroke, is a major problem that urgently needs to be solved.

The liver plays a crucial role in metabolism and is important for maintaining and regulating lipid and glucose levels as well as energy metabolism in the body. Extreme high temperatures may cause liver swelling, vacuolization, steatosis, and extensive centrilobular necrosis. Among the multiple organ injuries induced by heatstroke, acute liver injury and its further deterioration into acute liver failure are the main causes of death in heatstroke patients. Additionally, high-temperature exposure may lead to destruction of intestinal cell structure and function, consequently causing increased intestinal mucosal permeability and allowing bacteria and endotoxins from the intestinal tract to enter the bloodstream. As the core of the intestinal defense system, the intestinal mucosal barrier is mainly composed of the following four components: mechanical barrier (tightly connected epithelial cells), chemical barrier (secretions such as mucins and antimicrobial peptides), immune barrier (including immune cells and their secreted cytokines), and biological barrier (gut microbiota and their metabolites). These barriers work synergistically to maintain the stability of the intestinal environment and resist invasion by external harmful substances. The association between intestinal diseases and liver diseases, as well as the complex interactions among the intestine, liver, and microbial flora, is known as the “gut-liver axis” theory. The metabolic processes and disease progression of the liver are closely related to the gut microbiota. The liver can influence the composition and function of the gut microbiota through mechanisms such as secreting bile acids and immunoglobulin A. Currently, there are still no effective treatment measures for heatstroke complicated by acute liver injury. Therefore, seeking effective preventive interventions is an issue that urgently needs to be addressed, which has important public health significance.

For the lungs, extreme high temperatures often cause acute lung injury in the form of pulmonary edema and acute respiratory distress syndrome, with pathological manifestations including inflammatory response, oxidative stress response, and pulmonary dysfunction, characterized by diffuse alveolar damage caused by alveolar epithelium. The intestine and lungs have long been considered two closely connected organs. The concept of “the lungs and large intestine being internally and externally related” is an important theory in the holistic view of traditional Chinese medicine, which can be traced back to “Huangdi Neijing • Ling Shu • Ben Shu”: “the lungs connect with the large intestine, and the large intestine is the organ responsible for transmission”. From a histological and embryological perspective, the embryonic development of lung and intestinal tissues is homologous. Both originate from the primitive foregut and exhibit similar cell morphology, proliferation, and apoptosis during early embryonic development. Whether in a healthy or pathological state, the microorganisms and immune systems of the intestine and lungs are interconnected and interact with each other, hence referred to as the “gut-lung axis”. Currently, there is a lack of specific therapies for heatstroke complicated by acute lung injury, making the exploration of effective preventive interventions an urgent priority, which holds significant implications for the field of public health.

Extreme high temperatures may significantly increase the burden on the cardiovascular system, induce or exacerbate heart disease, and even lead to sudden death. Studies have found a significant correlation between heat waves and increased mortality from myocardial infarction. When the intestinal barrier is compromised, bacterial endotoxins (such as LPS) enter the bloodstream, triggering systemic low-grade inflammation, thereby promoting atherosclerosis and myocardial injury. The bidirectional interaction between the intestine and the heart, involving multiple levels such as gut microbiota, metabolites, immune inflammation, and neural regulation, is called the “gut-heart axis”. Currently, there is a lack of specific therapies for heatstroke complicated by acute myocardial injury, and the “heart-gut interaction” may become a new target for the prevention and treatment of cardiovascular diseases induced by extreme high temperatures.

Probiotics play an important role in treating immune inflammatory diseases by improving intestinal barrier function. Additionally, probiotic intervention mitigates metabolic disorders by regulating the balance of gut microbiota homeostasis. However, there is no technical inspiration or literature report regarding use of a six-strain probiotic for preventing severe heatstroke or heatstroke complicated by acute liver injury, lung injury, or myocardial injury in the prior art.

### SUMMARY

In view of the problems existing in the prior art, this disclosure aims to provide a six-strain probiotic for preventing severe heatstroke and use thereof, which are specifically implemented through the following technical solutions:
a six-strain probiotic for preventing severe heatstroke, including: Bacillus coagulans XY2, Lactobacillus plantarum KL100, Lactobacillus rhamnosus TR08, Lactobacillus helveticus TR05, Lactobacillus casei TR01, and Bifidobacterium longum TR17, where the probiotics are mixed in a viable count ratio of 1:1:1:1:1:1.

Use of the aforementioned six-strain probiotic in preparation of a drug for preventing and treating severe heatstroke.

Use of the aforementioned six-strain probiotic in preparation of a drug for enhancing intestinal mucosal barrier function compromised by severe heatstroke.

Use of the aforementioned six-strain probiotic in preparation of a drug for preventing severe heatstroke complicated by liver injury.

Further, the heatstroke complicated by liver injury refers to heatstroke complicated by acute liver injury.

Use of the aforementioned six-strain probiotic in preparation of a drug for preventing severe heatstroke complicated by lung injury.

Further, the heatstroke complicated by lung injury refers to heatstroke complicated by acute lung injury.

Use of the aforementioned six-strain probiotic in preparation of a drug for preventing severe heatstroke complicated by myocardial injury.

Further, the heatstroke complicated by myocardial injury refers to heatstroke complicated by acute myocardial injury.

The dosage form of the aforementioned six-strain probiotic is a liquid oral preparation, with an aqueous solution or milk selected as a solvent controlled at a temperature of below 37℃. The specific preparation steps are as follows: uniformly mixing the six-strain probiotic freeze-dried powder with the aqueous solution or milk at a temperature of below 37°C, where the “uniformly” means the system exhibits a homogeneous phase.

The dosage of the six-strain probiotic is 0.065 g/mL calculated based on the final concentration in mice, containing about 195 million CFU of viable probiotics.

Compared with the prior art, this disclosure has the following beneficial effects:
(1) this disclosure proposes for the first time new use of the six-strain probiotic in preparation of a drug for preventing severe heatstroke/heatstroke complicated by liver injury.
(2) The content of this disclosure clearly indicates that the six-strain probiotic preparation has an excellent effect in preventing severe heatstroke and can significantly improve the body's internal environmental homeostasis, enhance the body's tolerance threshold to high temperature through multiple mechanisms, and alleviate tissue injury caused by heat exposure. The preparation can effectively maintain the physiological balance of the body's core temperature regulation system and reduce the risk of severe heatstroke.
(3) Simultaneously, the six-strain probiotic preparation can also effectively protect the body from or alleviate heatstroke complicated by acute liver injury, and prevent heat stress-related abnormal liver function, serving as a potential drug for preventing and treating heatstroke complicated by liver injury. The dual protective effect of the six-strain probiotic in preventing severe heatstroke and its associated liver injury provides a new intervention strategy for the prevention and treatment of heat stress-related diseases.
(4) The six-strain probiotic preparation enhances intestinal mucosal barrier function, strengthens the body's ability to withstand high temperatures, and effectively prevents harmful substances from entering the bloodstream through the mucosal barrier. The technical content provided by this disclosure has significant application value in the field of drug development for severe heatstroke and its complications, and the breakthrough progress of this technology opens up new research directions for the clinical prevention and treatment of heat-related diseases as well as drug development.
(5) The six-strain probiotic preparation can effectively protect the body from or alleviate heatstroke complicated by acute lung injury, and prevent heat stress-related abnormal pulmonary function, serving as a potential drug for preventing and treating heatstroke complicated by lung injury. The dual protective effect of the six-strain probiotic in preventing severe heatstroke and its associated lung injury provides a new intervention strategy for the prevention and treatment of heat stress-related diseases.
(6) Simultaneously, the six-strain probiotic preparation can also effectively protect the body from or alleviate heatstroke complicated by acute myocardial injury, and prevent heat stress-related abnormal myocardial function, serving as a potential drug for preventing and treating heatstroke complicated by myocardial injury. The dual protective effect of the six-strain probiotic in preventing severe heatstroke and its associated myocardial injury provides a new intervention strategy for the prevention and treatment of heat stress-related diseases.

### BRIEF DESCRIPTION OF THE DRAWINGS

FIG. 1A-1B are graphs showing the effect of the six-strain probiotic preparation of this disclosure on changes in core body temperature in mice;

FIG. 2A-2C are graphs showing the effect of the six-strain probiotic preparation of this disclosure on liver morphology, serum alanine aminotransferase (ALT), and serum aspartate aminotransferase (AST) in mice;

FIG. 3A-3C are graphs showing the effect of the six-strain probiotic preparation of this disclosure on hepatic phosphatidate phosphatase (Lipin 1) in mice;

FIG. 4A-4C are graphs showing the effect of the six-strain probiotic preparation of this disclosure on intestinal mucosal barrier in mice;

FIG. 5A-5B are graphs showing the effect of the six-strain probiotic preparation of this disclosure on lung morphology in mice;

FIG. 6A-6B are graphs showing the effect of the six-strain probiotic preparation of this disclosure on lung inflammation in mice;

FIG. 7A-7D are graphs showing the effect of the six-strain probiotic preparation of this disclosure on oxidative stress in mouse lungs;

FIG. 8A-8C are graphs showing the effect of the six-strain probiotic preparation of this disclosure on pulmonary vascular barrier function in mice;

FIG. 9A-9B are graphs showing the effect of the six-strain probiotic preparation of this disclosure on heart morphology, lactate dehydrogenase (LDH), and creatine kinase isoenzyme (CK-MB) in mice;

FIG. 10A-10O are graphs showing the effect of the six-strain probiotic preparation of this disclosure on cardiac function in mice;

FIG. 11 is a graph showing the effect of the six-strain probiotic preparation of this disclosure on cardiac inflammation in mice; and

FIG. 12 is a graph showing the effect of the six-strain probiotic preparation of this disclosure on cardiac oxidative stress in mice (note: compared with the NC group, ^{*} P < 0.05, ^{**} P < 0.01, and ^{***} P < 0.001; compared with the HC group, ^{#} P < 0.05, ^{##} P < 0.01, and ^{###} P < 0.001. Significant differences were assessed using t-test, and results were expressed as mean ± SEM).

### DESCRIPTION OF THE EMBODIMENTS

This disclosure will be further described below in conjunction with specific embodiments.

The strains in the six-strain probiotic of this disclosure all come from the prior art and are commercially available, including:

Bacillus coagulans XY2, accession number: CGMCC NO. 33934, accessioned on March 24, 2025.

Lactobacillus plantarum KL100, accession number: CGMCC NO. 27192, accessioned on April 23, 2023.

Lactobacillus rhamnosus TR08, accession number: CCTCC NO: M2016548, accessioned on October 10, 2016.

Lactobacillus helveticus TR05, accession number: CCTCC NO: M2016569, accessioned on October 17, 2016.

Lactobacillus casei TR01, accession number: CCTCC NO: M2016567, accessioned on October 17, 2016.

Bifidobacterium longum TR17, accession number: CCTCC NO: M2017265, accessioned on May 15, 2017.

### Example 1: Preparation of Six-Strain Probiotic Solution and Model Intervention Protocol

The six-strain probiotic solution of this disclosure was prepared according to an oral dosage for humans of twice daily, 2 g per sachet, resulting in a daily dosage of 4 g for a standard body weight of 70 kg (4 g/70 kg). Based on the dosage per unit body weight, the equivalent dosage for a mouse (standard body weight 20 g) was 9.1 times that of a human. Based on this conversion factor, the daily dosage for mice was 0.52 g/kg, i.e., 0.52 mg/g. According to the gavage volume for mice being 8 μL/g, the amount of the six-strain probiotic in the oral preparation was calculated as 0.065 mg/μL (i.e., 0.065 g/mL) based on the final concentration in mice, meaning that 1 mL of aqueous solution (below 37℃) required 0.065 g of probiotic freeze-dried powder. The gavage volume for each mouse was obtained by multiplying the actual body weight of the mouse by the gavage volume, with the maximum gavage volume not exceeding 200 μL. The final probiotic content administered was approximately 195 million CFU of viable bacteria. During the process of optimizing the dosage of the six-strain probiotic, any dosage that can achieve the technical effects described in this disclosure falls within the technical solution of this disclosure and is within the scope of protection of this disclosure.

### Model Intervention Protocol

Eight-week-old healthy male C57BL/6N mice were purchased from Beijing Charles River Laboratory Animal Technologies Co. Ltd. (company license number: SCXK (Zhejiang) 2019-0001) and housed in Zhejiang Chinese Medical University Laboratory Animal Research Center. The mice were raised under conditions of 22±1℃, a 12 h:12 h light:dark cycle, and were given free access to food and water. After one week of adaptive feeding in an SPF-grade animal laboratory, the C57BL/6 mice were randomly divided into 4 groups as follows, with 6 mice in each group:
1) normal control group (NC);
2) control + preventive probiotic intervention group (CP);
3) heat control group (HC); and
4) heat + preventive probiotic intervention group (HP)

Probiotic intervention was administered to the mice one week before the beginning of heat exposure, once daily, by gavage at 21:00. The mice in the control group were gavaged with an equivalent volume of ddH₂O based on the body weight of the mice. The mice in the HC and HP groups were exposed to a high-temperature environment of 39.5±0.5℃. During the high-temperature exposure, the mice were deprived of water and food. The core body temperature of the mice was monitored with a rectal thermometer. When the core body temperature of the mice in the HC group reached a maximum value or a heat shock state was observed, the exposure was stopped. The mice in the HP group were exposed for the same duration as the mice in the HC group. The mice in the HP and HC groups, after the cessation of exposure, were transferred to an environment at 22±1℃ to recover for nine hours and were provided with water and feed.

Research Protocol: 1) based on the construction of the animal model in Example 1, after recovery for 9 h following high-temperature exposure, anesthesia was administered via intraperitoneal injection of sodium pentobarbital, and blood was collected from the inferior vena cava for determination of ALT and AST. The mice in the control group received the same treatment except for high-temperature exposure. 2) Detection of gene expression levels in liver tissue: using the above animal model, total RNA was extracted from liver samples with RNAiso Plus reagent, RNA was reverse transcribed into cDNA with a cDNA reverse transcription kit, and the mRNA level of Lipin 1 was detected with SYBR Green. 3) Detection of protein expression levels in liver tissue: using the above animal model, total protein was extracted from liver samples, the concentration was determined, and then the protein level of Lipin 1 was detected by Western Blot. 4) Detection of serum LPS, DAO, and D-Lactate levels by ELISA. 5) Detection of gene expression levels in lung tissue: using the above animal model, total RNA was extracted from lung samples with RNAiso Plus reagent, RNA was reverse transcribed into cDNA with a cDNA reverse transcription kit, and the mRNA levels of IL-6, Nf-κb, Cat, Sod1, Hmox-1, Nqo1, Occludin, Claudin-4, and Claudin-5 were detected with SYBR Green. 6) Based on the construction of the animal model in Example 1, after recovery for 9 h following high-temperature exposure, anesthesia was administered via intraperitoneal injection of sodium pentobarbital, and blood was collected from the inferior vena cava for determination of CK, LDH, and CK-MB. The mice in the control group received the same treatment except for high-temperature exposure. 7) Evaluation of cardiac function in the mice by echocardiography. 8) Detection of gene expression levels in heart tissue: using the above animal model, total RNA was extracted from heart samples with RNAiso Plus reagent, RNA was reverse transcribed into cDNA with a cDNA reverse transcription kit, and the mRNA levels of IL-6 and HO-1 were detected with SYBR Green.

### Example 2: Effect of the Six-Strain Probiotic Preparation on Changes in Core Body Temperature in Mice

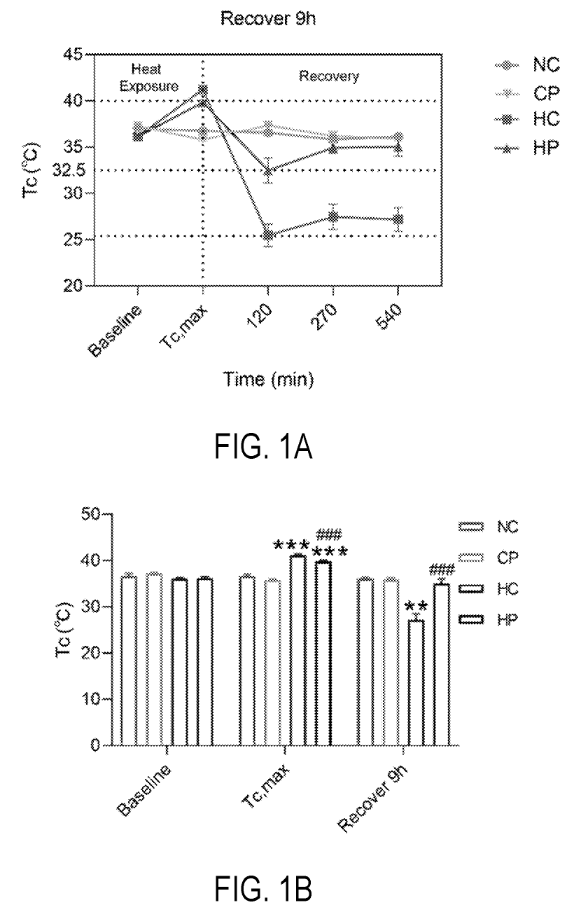

FIG. 1A-1B show the effect of the six-strain probiotic preparation of this disclosure on changes in core body temperature in mice. The results indicate that when the mice in the HC group reached a heat shock state (i.e., the core body temperature reached a maximum value), it was found that under the same conditions and within the same exposure time, the core body temperatures of the mice in the HC and HP groups were both significantly increased compared to the mice in the NC group; the maximum core body temperature of the mice in the HP group was significantly lower than that of the mice in the HC group (39.8℃ vs 41.2℃), demonstrating that the probiotics could prevent the phenomenon of excessive elevation of core body temperature under heat exposure. If the core body temperature becomes too high, it may lead to heat exhaustion or even death in mice. When the mice were removed from the high-temperature environment, the core body temperature of the high-temperature exposed mice began to decrease. The mice in the HC group all showed a rapid decrease in core body temperature, with a large drop within a short period, falling below 30℃, indicating a phenomenon of body temperature dysregulation. When the body temperature is too low, mice may exhibit unconsciousness, sluggish movement, and even shock, coma, or death. When the core body temperature of the mice in the HC group reached its minimum value, the minimum core body temperature of the mice in the HP group was significantly higher than that of the mice in the HC group (32.5℃ vs 25.5℃). The level of decrease in core body temperature of the mice in the HP group was significantly lower than that of the mice in the HC group. Even when dropped to the lowest temperature, it remained within the normal body temperature regulation range, without exhibiting severe body temperature dysregulation, and no symptoms related to severe heatstroke were observed (FIG. 1A). Furthermore, during the recovery period, the core body temperature was able to return to normal levels quickly. The core body temperature of the mice in the HP group had already returned to a normal level (35.1℃), while that of the mice in the HC group (27.2℃) had not returned to a normal level (FIG. 1B).

From the above results, it can be seen that the six-strain probiotic preparation can effectively regulate the dynamic changes in body temperature of high-temperature exposed mice. The specific manifestations are as follows: inhibiting the excessively rapid increase in body temperature under high-temperature exposure, significantly delaying the rate of core body temperature rise, enhancing the body's resistance to high temperatures, promoting the recovery of body temperature homeostasis, and accelerating thermoregulatory function during the recovery period. The six-strain probiotic preparation can also significantly mitigate the level of decrease in core body temperature during the recovery period in high-temperature exposed mice, restore the core body temperature of mice within a short period, and prevent the occurrence of severe body temperature dysregulation in high-temperature exposed mice. When the core body temperature of mice falls below normal levels, beyond the self-regulatable range of the body, it can affect metabolism, the central nervous system, the circulatory system, and liver function, and in severe cases, can be life-threatening. Therefore, the experimental results demonstrate that the six-strain probiotic preparation can prevent severe heatstroke in mice, improve the body's heat adaptation ability, reduce the probability of severe heatstroke in mice, and improve physiological function during the recovery period. Thus, when the six-strain probiotic preparation is used in the preparation of a drug for preventing severe heatstroke, it will have a good effect in preventing severe heatstroke and improving the overall functional status of patients during the post-exposure recovery period.

### Example 3: Effect of the Six-Strain Probiotic Preparation on Liver Morphology, Serum Alanine Aminotransferase (ALT), and Serum Aspartate Aminotransferase (AST) in Mice

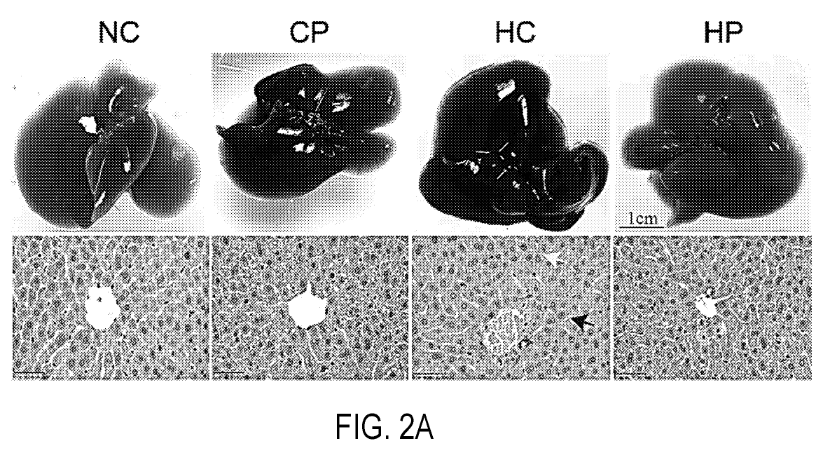

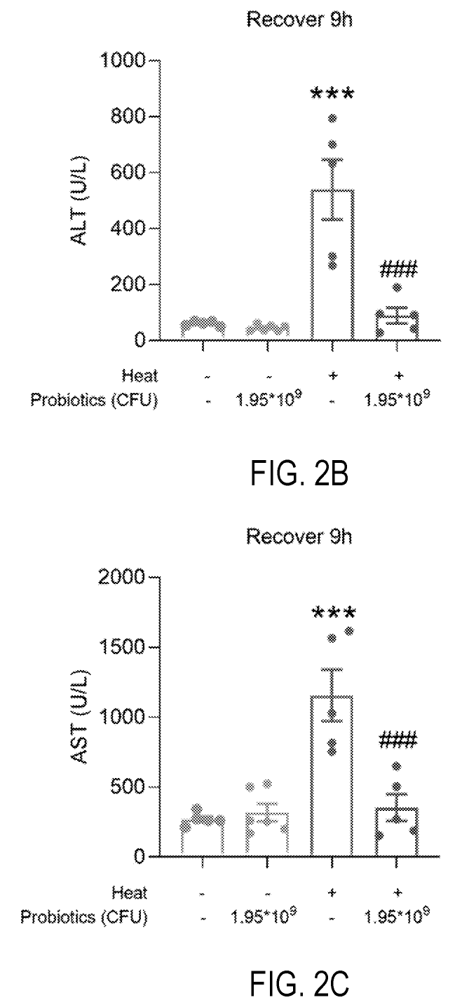

From the liver tissue morphology and staining of the mice in the four groups NC, CP, HC, and HP, it could be observed that the livers of the mice in the NC and CP groups were pink or dark red with a smooth surface; and the livers of the mice in the HC group had a darkened color, dark red tissue, and a disordered and irregular surface structure. The liver color of the mice in the HP group was similar to that of the mice in the NC group. In the NC and CP groups, the hepatocytes of the mice were radially arranged in cord-like structures centered around the central vein, forming hepatic cell cords, with irregular spaces between the hepatic cell cords being the sinusoids; and the liver tissue morphology of the mice in the HC group was abnormal, showing sinusoidal congestion and vacuolar degeneration. Preventive probiotic intervention significantly ameliorated the above phenomena (FIG. 2A).

ALT is mainly present in the hepatocyte cytoplasm, and its level in serum is normally very low. When hepatocytes are damaged, cell membrane permeability increases, and intracellular ALT is released into the blood. ALT is the most sensitive indicator for detecting liver function damage. A low serum ALT level indicates that liver function has not been significantly impaired. Compared with the control group, serum ALT was significantly increased in the mice from the heat exposure group, and preventive probiotic intervention significantly reduced the serum ALT level (FIG. 2B).

Most AST is present in liver mitochondria, and its level in serum is normally very low. When severe liver injury occurs, often accompanied by mitochondrial membrane damage, AST is released into the blood. AST reflects the severity of hepatocyte damage. A low serum AST level indicates that liver function has not been significantly impaired. Similarly, compared with the control group, serum AST was significantly increased in the mice from the heat exposure group, and preventive probiotic intervention significantly reduced the serum AST level (FIG. 2C). The above results indicate that preventive probiotic intervention can significantly improve liver morphology and alleviate liver injury in mice exposed to extreme high temperatures.

### Example 4: Effect of the Six-Strain Probiotic Preparation on Hepatic Phosphatidate Phosphatase (Lipin 1) in Mice

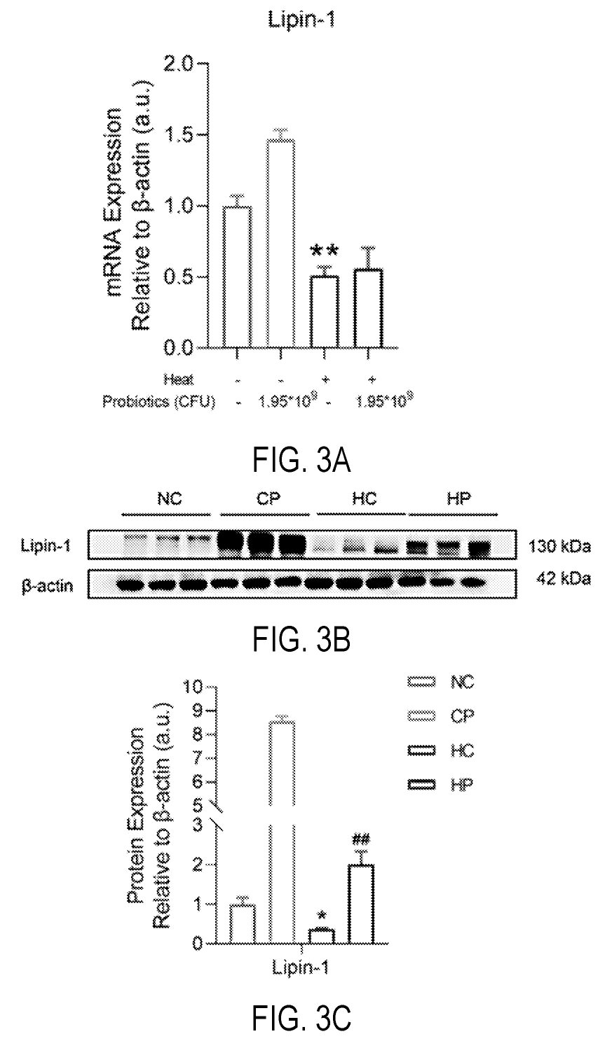

The experimental results indicate that, compared with the control group, the mRNA expression level of Lipin 1 in the livers of the mice in the heat exposure group was significantly decreased, and preventive probiotic intervention up-regulated the mRNA expression of Lipin 1 to a certain extent (FIG. 3A). Compared with the NC group, the protein expression level of Lipin 1 in the livers of the mice in the HC group was significantly decreased; compared with the HC group, the protein expression level of Lipin 1 in the livers of the mice in the HP group was significantly increased (FIG. 3B-3C). The Lipin 1 gene was identified in 2001 as a key gene closely related to lipid deposition, and the Lipin 1 protein it encodes is a member of the Lipin family. In the cytoplasm, Lipin 1 catalyzes the dephosphorylation of phosphatidic acid to generate diacylglycerol through its phosphatidate phosphatase activity. This process is a key step in triglyceride synthesis and is crucial for lipid storage. Down-regulation of Lipin 1 expression promotes an increase in diacylglycerol level in the liver, thereby interfering with insulin receptor signaling pathways, ultimately leading to lipid metabolism disorders and lipid deposition. From the above experimental results, it can be seen that under high-temperature exposure conditions, the expression of Lipin 1 in the livers of the mice in the high-temperature exposure group was significantly down-regulated, suggesting a disorder in liver lipid metabolism function; meanwhile, compared with the high-temperature exposure group, the expression of Lipin 1 in the livers of the mice was significantly increased under the action of the probiotic. The above results indicate that preventive probiotic intervention can significantly enhance the expression of Lipin 1 in the livers of mice exposed to extreme high temperatures and improve liver lipid metabolism.

### Example 5: Effect of the Six-Strain Probiotic Preparation on Intestinal Mucosal Barrier

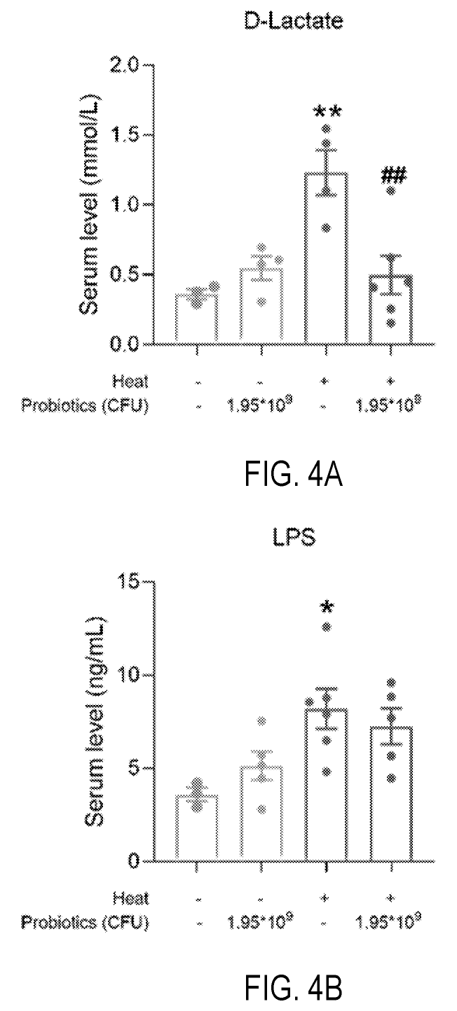

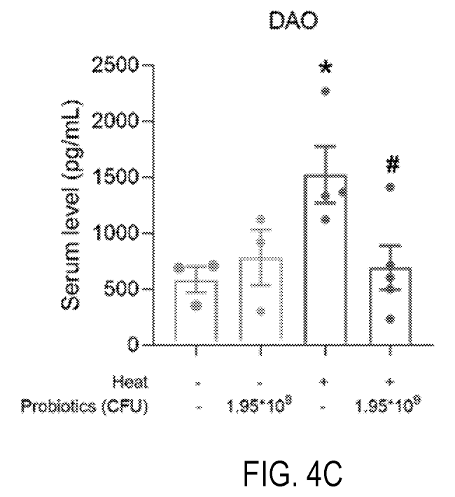

Compared with the control group, the serum D-Lactate, LPS, and DAO levels in the mice of the heat exposure group were all significantly increased; compared with the heat exposure group, preventive probiotic intervention resulted in a significant decrease in the serum D-Lactate level (FIG. 4A), a decreasing trend in the serum LPS level (FIG. 4B), and a significant decrease in the serum DAO level (FIG. 4C) in the heat-exposed mice. The serum D-Lactate, endotoxin (LPS), and diamine oxidase (DAO) levels are important indicators for evaluating the integrity of the intestinal barrier. DAO is mainly derived from small intestinal mucosal epithelial cells. An increase in its level typically reflects structural damage to the intestinal mucosal epithelial cells and functional disruption of the intestinal mucosal mechanical barrier. D-Lactate, a major metabolite of the gut microbiota, serves as a hallmark for impaired intestinal mucosal integrity and increased permeability when its level is elevated. LPS is an important component of the outer membrane of Gram-negative bacteria and is usually enriched in the intestinal tract. As the largest source of endotoxin in the body, LPS does not easily enter the bloodstream when the intestinal barrier function is intact. However, when the intestinal barrier is disrupted, LPS can penetrate the intestinal mucosa and enter the blood, thereby inducing endotoxemia and systemic inflammatory responses. From the above experimental results, it can be seen that under high-temperature exposure conditions, the serum D-Lactate, LPS, and DAO levels in the mice of the high-temperature exposure group were all significantly increased, indicating impaired intestinal mucosal integrity, increased permeability, and the release of endotoxin into the bloodstream, leading to systemic inflammation. Meanwhile, compared with the high-temperature exposure group, under the action of the probiotic, the serum D-Lactate, LPS, and DAO levels, which are related to mucosal barrier function, were significantly reduced in the mice. This demonstrates that preventive probiotic intervention can significantly enhance the intestinal mucosal barrier function in mice exposed to extreme high temperatures.

### Example 6: Effect of the Six-Strain Probiotic Preparation on Lung Morphology in Mice

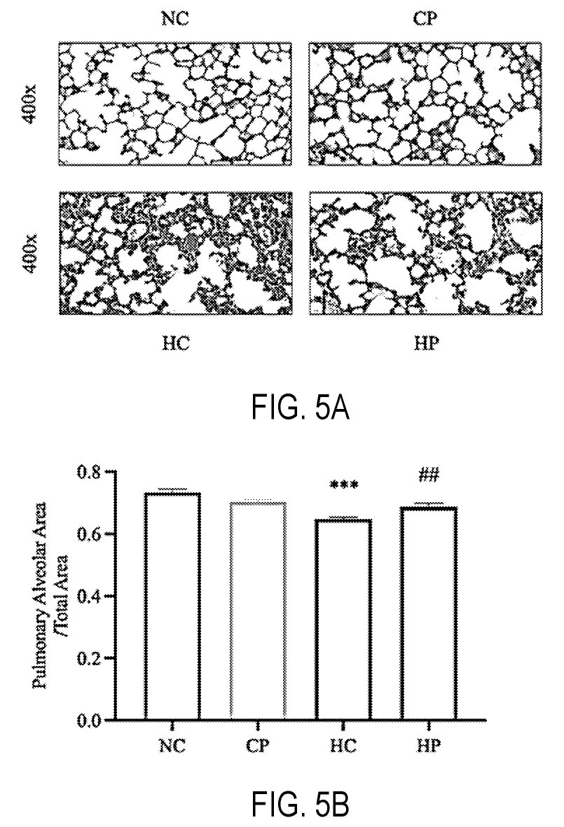

Compared with the control group, substantial alveolar collapse appeared in the lungs of heat-exposed mice, and the alveolar surface area was significantly reduced, suggesting that heat exposure led to atelectasis. Compared with the heat exposure group, preventive probiotic intervention significantly alleviated alveolar collapse and also significantly increased the alveolar surface area in the lungs of heat-exposed mice (FIG. 5A). Semi-quantitative results showed that the pulmonary alveolar area/total area in the lungs of the heat-exposed mice was significantly lower than that of the control group; whereas the pulmonary alveolar area/total area in the lungs of the mice with preventive probiotic intervention was significantly higher than that of the heat exposure group. The above results indicate that probiotic intervention can effectively alleviate atelectasis caused by heat exposure (FIG. 5B).

### Example 7: Effect of the Six-Strain Probiotic Preparation on Lung Inflammation in Mice

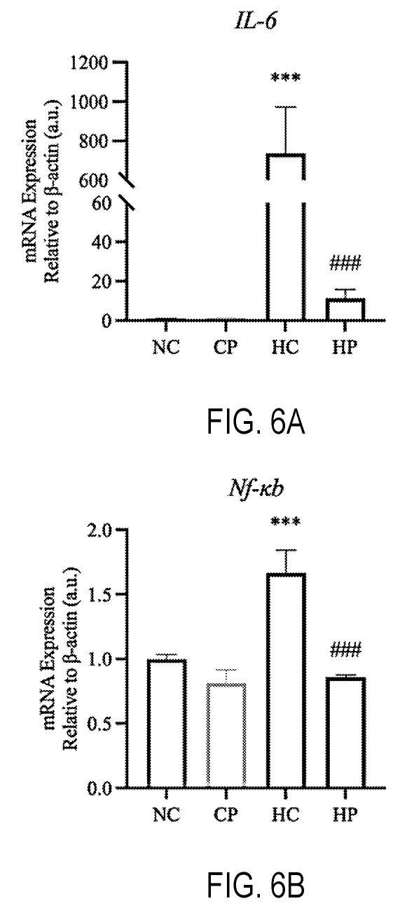

Compared with the control group, the mRNA levels of inflammatory factors IL-6 and Nf-κb in the lungs of heat-exposed mice were significantly up-regulated; compared with the heat exposure group, preventive probiotic intervention significantly inhibited the mRNA levels of IL-6 (FIG. 6A) and Nf-κb (FIG. 6B) in the lungs of heat-exposed mice. This indicates that preventive probiotic intervention can significantly ameliorate lung inflammation in mice caused by heat exposure.

### Example 8: Effect of the Six-Strain Probiotic Preparation on Oxidative Stress in Mouse Lungs

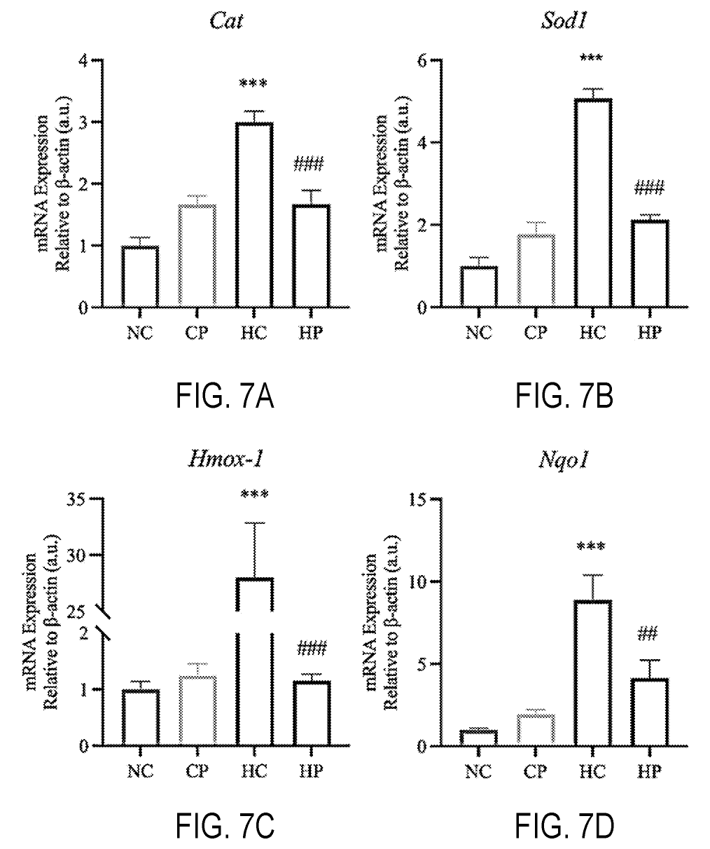

Detection of the expression levels of antioxidant enzymes related to oxidative stress showed that the mRNA levels of the antioxidant enzymes Cat, Sod1, Hmox-1, and Nqo1 in the lungs of heat-exposed mice were significantly higher than those in the control group; compared with the heat exposure group, preventive probiotic intervention significantly down-regulated the mRNA levels of Cat (FIG. 7A), Sod1 (FIG. 7B), Hmox-1 (FIG. 7C), and Nqo1 (FIG. 7D). Cat, Sod1, Hmox-1, and Nqo1 are all key antioxidant enzymes that collaboratively maintain cellular redox homeostasis and resist oxidative stress damage by decomposing hydrogen peroxide, scavenging superoxide, degrading pro-oxidant heme, and reducing quinones, respectively. The above results indicate that heat exposure disrupted the redox homeostasis in mouse lungs, causing a compensatory up-regulation of antioxidant enzyme expression; whereas preventive probiotic intervention was able to alleviate the redox imbalance caused by heat exposure and restore antioxidant enzyme levels.

### Example 9: Effect of the Six-Strain Probiotic Preparation on Pulmonary Vascular Barrier Function in Mice

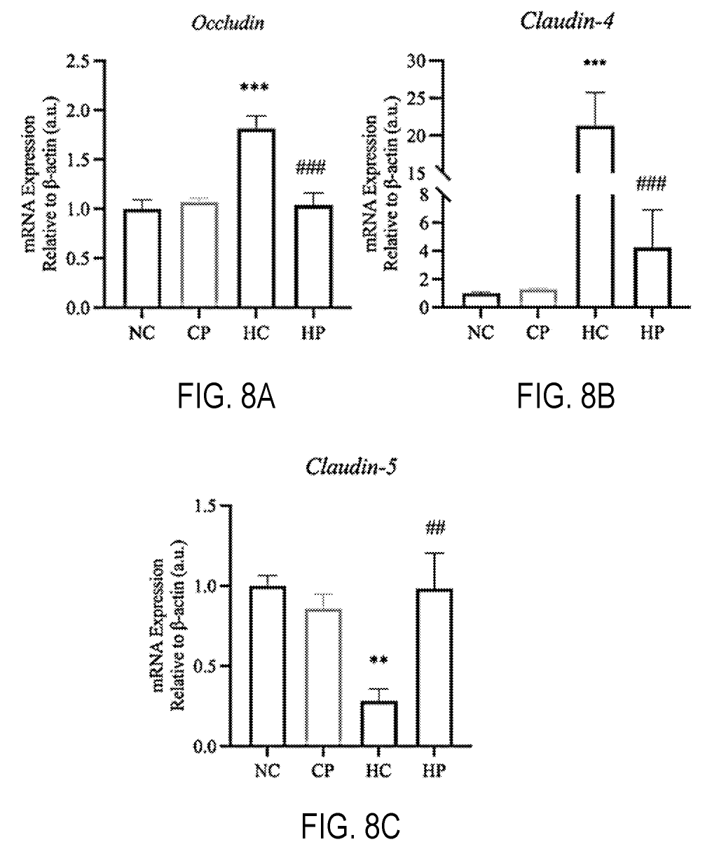

Compared with the control group, the mRNA levels of the pulmonary vascular barrier-related channel proteins Occludin and Claudin-4 were significantly increased in heat-exposed mice, while the mRNA level of Claudin-5 was significantly decreased; compared with the heat exposure group, preventive probiotic intervention significantly reduced the mRNA levels of Occludin (FIG. 8A) and Claudin-4 (FIG. 8B) in mouse lungs, and increased the mRNA level of Claudin-5 (FIG. 8C). The tight junction proteins Occludin, Claudin-4, and Claudin-5 in the pulmonary vascular barrier jointly maintain the integrity and permeability of the alveolar epithelium primarily by participating in the formation and regulation of tight junctions, thereby ensuring normal gas exchange and fluid balance in the lungs. Lung injury can lead to decreased expression of the core tight junction protein Claudin-5, and the body may up-regulate the expression of Occludin and Claudin-4 to repair the damaged tight junction structure and maintain barrier integrity. The above results indicate that high-temperature exposure can disrupt the tight junction structure of the pulmonary vascular barrier in mice, impairing the “molecular sieve” function of the vascular endothelial barrier, consequently leading to up-regulated expression of Occludin and Claudin-4 to repair the damaged barrier; whereas preventive probiotic intervention can up-regulate the expression of Claudin-5, alleviate inflammation and oxidative stress levels, protect the integrity of the pulmonary vascular barrier, and restore the normal expression levels of Occludin and Claudin-4.

### Example 10: Effect of the Six-Strain Probiotic Preparation on Heart Morphology, Lactate Dehydrogenase (LDH), and Creatine Kinase Isoenzyme (CK-MB) in Mice

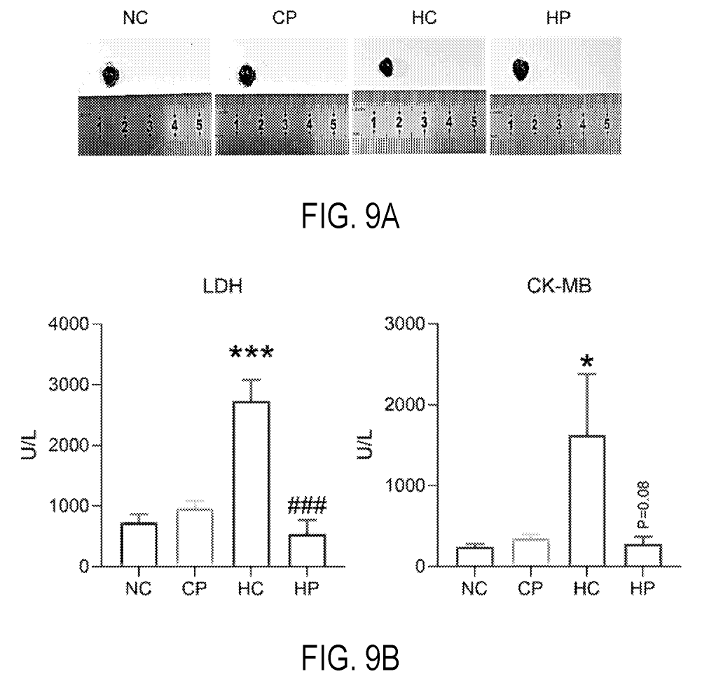

Analysis of serum cardiac enzymes revealed that, compared with the control group, the hearts of the mice in the heat exposure group were smaller in size and darker in color; compared with the heat exposure group, following preventive probiotic intervention, the hearts were larger in size and lighter in color, approaching normal cardiac morphology (FIG. 9A). Compared with the control group, LDH was significantly up-regulated in heat-exposed mice; compared with the heat exposure group, preventive probiotic intervention down-regulated the LDH level (FIG. 9B). Compared with the control group, the CK-MB level was elevated in heat-exposed mice; compared with the heat exposure group, preventive probiotic intervention down-regulated the CK-MB level (FIG. 9C). LDH is an important cellular metabolic enzyme that is highly sensitive to tissue injury and can serve as an auxiliary diagnostic indicator for the later stage of acute myocardial infarction. CK-MB is one of the sensitive indicators for myocardial tissue injury. When myocardial tissue is severely injured, CK-MB is released into the blood, and CK-MB in the serum becomes an important criterion for diagnosing acute myocardial infarction. The above results indicate that preventive probiotic intervention can significantly alleviate cardiac morphology changes and myocardial injury in heat-exposed mice.

### Example 11: Effect of the Six-Strain Probiotic Preparation on Cardiac Function in Mice

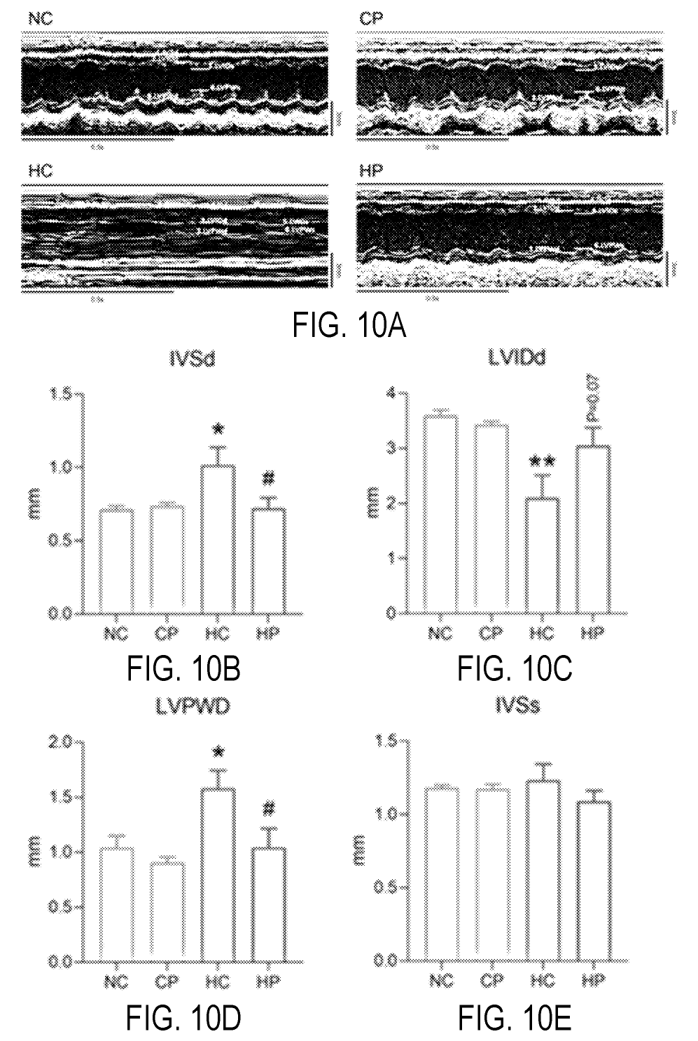

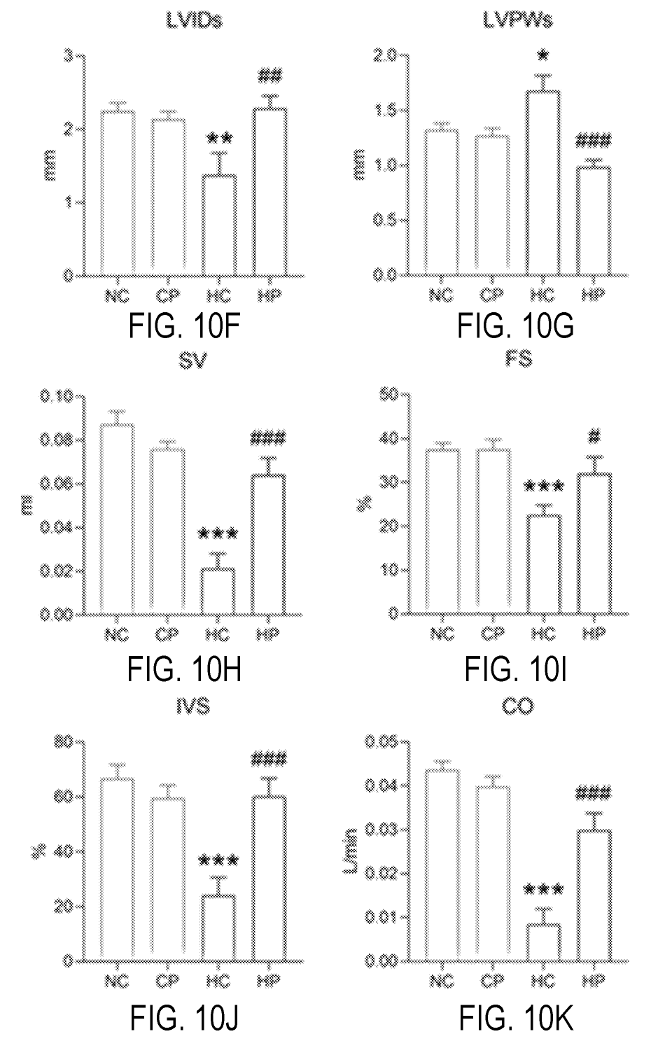

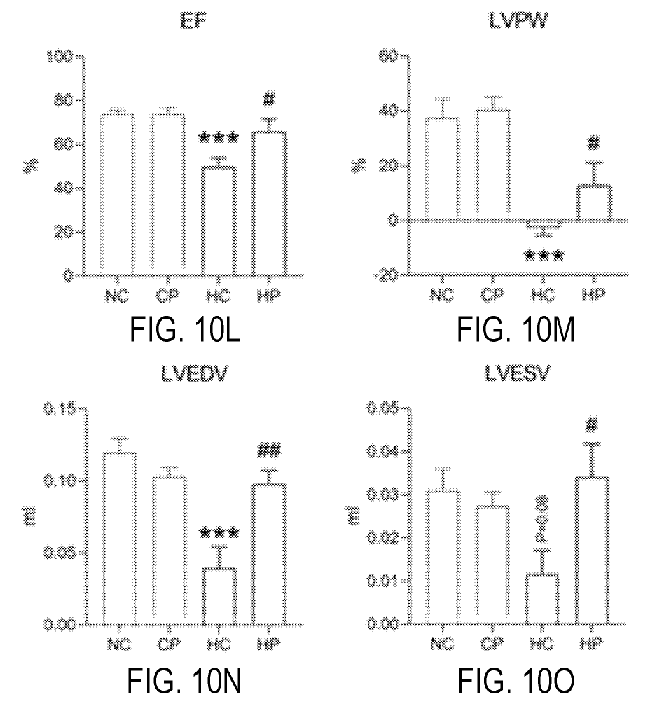

Compared with the control group, the cardiac volume and function levels of the mice in the heat exposure group were significantly decreased, and the cardiac structure was significantly altered; compared with the heat exposure group, preventive probiotic intervention significantly improved the cardiac structure and function of heat-exposed mice and increased the volume level (FIG. 10A).

From the above experimental results, it can be seen that compared with the control group, the interventricular septal thickness at end-diastole (IVSd), left ventricular posterior wall thickness at end-diastole (LVPWD), and left ventricular free wall thickness at end-systole (LVPWs) were significantly increased in the mice of the heat exposure group, while the left ventricular internal diameter at end-diastole (LVIDd) and left ventricular internal diameter at end-systole (LVIDs) were significantly decreased, and the interventricular septal thickness at end-systole (IVSs) showed no significant change; compared with the heat exposure group, in the probiotic intervention group, IVSd was significantly decreased (FIG. 10B), LVIDd showed an increasing trend (FIG. 10C), LVPWD was significantly decreased (FIG. 10D), IVSs showed no significant change (FIG. 10E), LVIDs was significantly increased (FIG. 10F), and LVPWs was significantly decreased (FIG. 10G). Among the structural indicators, IVSd, LVIDd, LVPWD, IVSs, LVIDs, and LVPWs are important indicators for evaluating cardiac structure. An increase in IVSd and IVSs levels indicates an increase in interventricular septal thickness, serving as an important indicator for evaluating myocardial hypertrophy. A decrease in LVIDd and LVIDs levels indicates a decrease in left ventricular size, which can serve as a hallmark of cardiac shrinkage. LVPWD and LVPWs represent the thickness of the left ventricular posterior wall and free wall, respectively, and can assist in evaluating the myocardial state. After extreme high-temperature exposure, cardiac shrinkage and myocardial hypertrophy occurred; preventive probiotic intervention was able to ameliorate the cardiac structure in the mice after extreme high-temperature exposure.

In terms of functional indicators, compared with the control group, the functional indicators of the mice in the heat exposure group, including stroke volume (SV), left ventricular fractional shortening (FS), interventricular septal thickening fraction during systole (IVS), cardiac output (CO), ejection fraction (EF), and left ventricular posterior wall thickening fraction during systole (LVPW), were significantly decreased; compared with the heat exposure group, after probiotic intervention, SV (FIG. 10H), FS (FIG. 10I), IVS (FIG. 10J), CO (FIG. 10K), EF (FIG. 10L), and LVPW (FIG. 10M) were significantly increased. SV refers to the volume of blood ejected in one heartbeat. The FS and EF levels are important indicators for evaluating left ventricular systolic function, reflecting myocardial contractility. CO refers to the cardiac output per minute, and its level can measure the pumping function of the heart. The IVS and LVPW levels reflect the degree of thickening of the interventricular septum and left ventricular posterior wall during systole, indicating myocardial contractile performance. When the structural level of the heart changes, it first enters a compensatory phase with decreased internal diameter and myocardial thickening; when the injury continues, it enters a decompensatory phase with reduced volume and decreased function, ultimately developing into heart failure. The decrease in the above functional indicators in the heat-exposed mice suggests impaired cardiac function in the heat-exposed mice. Preventive probiotic intervention was able to increase the levels of these indicators, thereby improving the cardiac function in the mice after extreme high-temperature exposure.

In terms of volume indicators, compared with the control group, the left ventricular end-diastolic volume (LVEDV) and left ventricular end-systolic volume (LVESV) of the heat-exposed mice were significantly decreased; compared with the heat exposure group, after probiotic intervention, LVEDV (FIG. 10N) and LVESV (FIG. 10O) were significantly increased. Among the volume indicators, the LVEDV and LVESV levels represent the volume of the left ventricle at the end of diastole and systole, respectively, reflecting cardiac preload and pumping function. The volume indicators of the heat-exposed mice were significantly decreased, and preventive probiotic intervention was able to increase the levels of the volume indicators, thereby improving the cardiac volume in the mice after extreme high-temperature exposure. In summary, preventive probiotic intervention was able to ameliorate the cardiac structure and function in the mice after extreme high-temperature exposure.

### Example 12: Effect of the Six-Strain Probiotic Preparation on Cardiac Inflammation in Mice

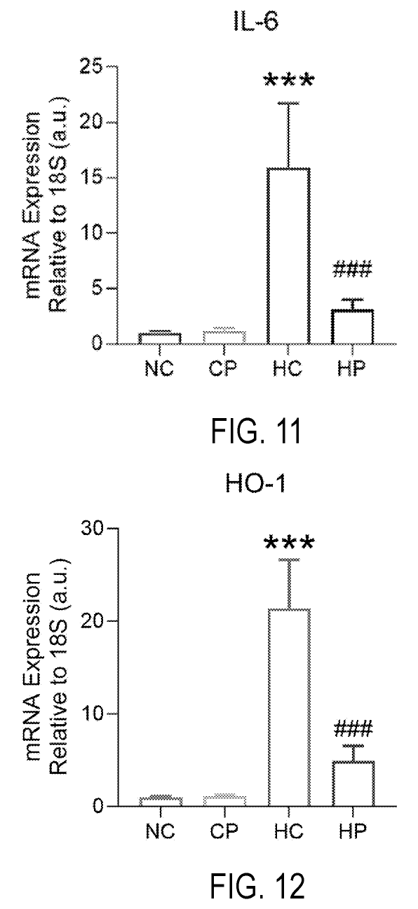

Compared with the control group, interleukin-6 (IL-6) was significantly up-regulated after extreme high-temperature exposure; compared with the heat exposure group, probiotic intervention significantly down-regulated the IL-6 level (FIG. 11). IL-6 is an inflammatory factor involved in the pathological processes of various infectious and inflammatory diseases, and an increase in its level indicates an elevated level of inflammation in the body. Preventive probiotic intervention was able to down-regulate the IL-6 level, significantly ameliorating cardiac inflammation in the heat-exposed mice.

### Example 13: Effect of the Six-Strain Probiotic Preparation on Cardiac Oxidative Stress in Mice

Compared with the control group, heme oxygenase-1 (HO-1) was significantly up-regulated after extreme high-temperature exposure; compared with the heat exposure group, probiotic intervention significantly down-regulated the HO-1 level (FIG. 12). HO-1 is an important antioxidant enzyme. The mRNA and protein expression of HO-1 can be up-regulated in response to oxidative stress and cellular damage, and HO-1 is also considered to be involved in vascular protection. The HO-1 level in cardiac tissue was significantly increased after heat exposure, while preventive probiotic intervention was able to alleviate the redox imbalance caused by heat exposure and restore the level of this antioxidant enzyme.

What is claimed is:

1. A six-strain probiotic for preventing severe heatstroke, comprising: Bacillus coagulans XY2, Lactobacillus plantarum KL100, Lactobacillus rhamnosus TR08, Lactobacillus helveticus TR05, Lactobacillus casei TR01, and Bifidobacterium longum TR17, wherein the probiotics are mixed in a viable count ratio of 1:1:1:1:1:1.

2. Use of the six-strain probiotic of claim 1 in preparation of a drug for preventing and treating severe heatstroke.

3. Use of the six-strain probiotic of claim 1 in preparation of a drug for enhancing intestinal mucosal barrier function compromised by severe heatstroke.

4. Use of the six-strain probiotic of claim 1 in preparation of a drug for preventing severe heatstroke complicated by liver injury.

5. The use of claim 4, wherein the heatstroke complicated by liver injury refers to heatstroke complicated by acute liver injury.

6. Use of the six-strain probiotic of claim 1 in preparation of a drug for preventing severe heatstroke complicated by lung injury.

7. The use of claim 6, wherein the heatstroke complicated by lung injury refers to heatstroke complicated by acute lung injury.

8. Use of the six-strain probiotic of claim 1 in preparation of a drug for preventing severe heatstroke complicated by myocardial injury.

9. The use of claim 8, wherein the heatstroke complicated by myocardial injury refers to heatstroke complicated by acute myocardial injury.
